(19) United States
(12) Patent Application Publication
(10) Pub. No.: US 2023/0003745 A1
(43) Pub. Date: Jan. 5, 2023

(54) METHOD FOR MAKING A FINDING FOR THE FUNCTIONALITY OF AN ANOREXIGENIC SIGNAL PATH FOR A PATIENT

(71) Applicant: Labor Berlin - Charité Vivantes Services GmbH, Berlin (DE)

(72) Inventors: Tina BUCHHOLZ, Berlin (DE); Nancy KRÜGER, Brandenburg (DE); Oliver BLANKENSTEIN, Berlin (DE); Peter KÜHNEN, Berlin (DE); Falko BÖHRINGER, Berlin (DE)

(73) Assignee: Labor Berlin - Charité Vivantes Services GmbH, Berlin (DE)

(21) Appl. No.: 17/780,588

(22) PCT Filed: Nov. 27, 2020
(86) PCT No.: PCT/EP2020/083643

§ 371 Date: May 27, 2022

(30) Foreign Application Priority Data
Nov. 29, 2019 (DE) 10 2019 218 598.3

(51) Int. Cl. G01N 33/74

(52) U.S. Cl. CPC G01N 33/74; G01N 2800/04

(57) ABSTRACT

The present invention relates to a method of providing a FAS finding (30) for the functionality of an anorexigenic signal path for a patient, comprising the steps: providing a sample matrix (10) of a body substance of the patient, determining at least one first FAS indicator (11) from the sample matrix (10), determining at least one second FAS indicator (12) from the sample matrix (10), wherein the at least one second FAS indicator (12) is different from the at least one first FAS indicator (11), and generating the FAS finding (30) using an indicator spectrum (20) comprising the at least one first FAS indicator (11) and the at least one second FAS indicator (12). The invention also relates to an analysis device (100) for providing a FAS finding (30) with a computer program product according to the invention (90) and a storage means (80) with a computer program product (90) according to the invention stored thereon.

The present invention relates to a method of providing a finding for the functionality of an anorexigenic signal path for a patient or subject. The invention further relates to a computer program product for carrying out such a method and a storage means with a computer program product of the generic type for providing such a finding stored thereon. The invention also relates to an analysis device for generating such a finding.

According to current studies, it is assumed that one in three people worldwide is over-weight or obese. In cases of excess weight which can be harmful to health, one speaks of obesity. Obesity is now regarded as a chronic disease which is associated with an impaired quality of life and a high risk of secondary diseases. Not only can those affected suffer from physical consequences, they are often victims of discrimination within the population.

The causes of obesity vary. In addition to excessive calorie intake resulting from unhealthy diets, obesity can also be caused or at least influenced by genetic factors.

According to the International Classification of Diseases and Related Health Problems, obesity is classed under the endocrine, nutritional and metabolic diseases.

Body mass index (BMI) serves as a rough measure for determining obesity. The BMI is calculated by dividing the body weight in kilograms by the height in metres squared. If the BMI is over 30 kg/m², this is classed as obesity according to the World Health Organisation. The BMI also allows classification into different degrees of obesity. Thus, with a BMI of between 30 and 34.9 one speaks of grade I obesity, with a BMI of between 35 and 39.9 of grade II obesity and with a BMI of 40 and more of grade III obesity or obesity permagna or morbid obesity. However, BMI is only a rough guide value. A BMI-based diagnosis is not sufficient for a targeted treatment of affected patients. In particular, the BMI does not allow a distinction to be made between excess weight due to a disturbed energy balance and an obesity which may be caused by genetic factors. Other approaches to the diagnosis and subsequent treatment of obesity known in the prior art have not as yet delivered the desired success.

The object of the present invention is to take into account at least partially the problems described above. In particular, it is the object of the present invention to create a method, a computer program product, a storage means and an analysis device for recognising the causes of genetic obesity.

The above object is achieved by the claims. In particular, the above object is achieved by the method according to claim 1, the computer program product according to claim 12, the storage means according to claim 13 and the analysis device according to claim 14. Further advantages of the invention emerge from the dependent claims, the description and the drawings. Features and details which are described in connection with the method naturally also apply in connection with the computer program product according to the invention, the storage means according to the invention, the analysis device according to the invention and vice versa, so that with regard to disclosure, reference is or can always made to the individual aspects of the invention.

According to a first aspect of the present invention, a method of providing a FAS finding for the functionality of an anorexigenic signal path for a patient is provided. The method comprises the following steps:
providing a sample matrix of a body substance of the patient,
determining at least one first FAS indicator from the sample matrix,
determining at least one second FAS indicator from the sample matrix, wherein the at least one second FAS indicator is different from the at least one first FAS indicator, and
generating the FAS finding using an indicator spectrum comprising the at least one first FAS indicator and the at least one second FAS indicator.

FAS is to be understood here as an abbreviation for the functionality of an anorexigenic signal path. Accordingly, the FAS finding is to be understood as a finding regarding the functionality of an anorexigenic signal path. In other words, the FAS finding can be understood as an assessment of the function of an anorexigenic signal path. A FAS indicator is, accordingly, to be understood as an indicator for assessing or diagnosing the functionality of an anorexigenic signal path. The FAS finding can therefore be arrived at with the help of FAS indicators. Depending on the functionality of the anorexigenic signal path, the patient can achieve the desired feeling of satiety at different times, which helps them maintain a regulated diet. If the anorexigenic signal pathway is disturbed, this can lead to the patient not experiencing any or only a reduced feeling of satiety and thus being more at risk of obesity than a person whose anorexigenic signal pathway is undisturbed or whose anorexigenic signal path functions as desired.

In the context of the present invention, it was recognised that the finding regarding the functionality of the anorexigenic signal path reliably allows direct conclusions to be drawn regarding an obesity, in particular a genetic obesity. Furthermore, it was recognised that the function of the signal path can be assessed on the basis of different indicators. This finding has been further developed to the effect that the FAS finding is generated using an indicator spectrum comprising the various FAS indicators. The joint consideration of a plurality of different FAS indicators allows a significant gain in information or a correspondingly high accuracy of the FAS finding to be achieved. The indicator spectrum can therefore be understood to mean a collection of different FAS indicators. Accordingly, according to the invention a plurality of different FAS indicators are considered jointly.

It is generally difficult to achieve a reliable diagnosis on the basis of a single indicator chemically in the laboratory, because often the technical detection limit of a detection method lies very close to a lower end of the normal range and as a rule there is little space available below this for assessing a clearly pathological situation. According to the invention, a better or more accurate result is now derived from the overall view of the indicator spectrum than would be possible on the basis of a sum of individual findings.

With knowledge of a predefined symptomatology and further diagnostic measures, for example involving imaging, clinical symptoms or genetics, patterns characteristic of individual diagnoses can be identified from a larger number of analyses on the basis of the indicator spectrum. These can then be applied to the patient, without detailed knowledge of the broader diagnostic measures, to predict the diagnosis, the further progression of the condition or, for example, also the response to certain drug treatments. These patterns contain laboratory results which are conspicuous, not only in the sense of classical laboratory analysis.

The at least one first FAS indicator differs, in particular in its nature, from the at least one second FAS indicator. That is to say, the at least one first FAS indicator differs not only in amount and/or scope from the at least one second FAS indicator. That the indicator spectrum comprises the at least one first FAS indicator and the at least one second FAS indicator is to be understood to the effect that the respective FAS indicator can basically comprise an unlimited number of different FAS indicators. Thus, the indicator spectrum can also comprise three or more FAS indicators which are in each case different from each other.

The generation of a FAS finding can be understood to mean a finding under which in the present case medically relevant physical or psychological phenomena, circumstances, changes and/or conditions of the patient relating to the functionality of their anorexigenic signal path are collected. Providing the sample matrix can be understood to mean providing at least one sample matrix. Patient can also be understood to refer to a test subject.

According to a further development of the present invention it is possible that, in a method, the at least one first FAS indicator comprises an MSH concentration, in particular an α-MSH concentration, in the sample matrix. Neuronal α-MSH is formed in the hypothalamus and anterior pituitary lobe from the proprotein proopiomelanocortin (POMC). Hypothalamic α-MSH plays a central role in weight regulation in that, on the basis of signals from the periphery, the fat content of the body and the stomach filling lead to the release of α-MSH, which conveys the feeling of satiety via the central melanocortin receptors. Mutations in the POMC gene can lead to a deficiency of the proprotein and thus of the α-MSH. Thus, due to the interrupted signal chain, no satiety signal can be generated. This can lead to uncontrolled, greatly increased food intake and thus to extreme obesity. In experiments conducted in connection with the present invention, it was shown that satiety is associated with an increase in the α-MSH concentration, in particular in the cerebrospinal fluid. Defects in this central α-MSH signal transmission have so far only been diagnosed on the basis of the underlying genetic defects, on the one hand because the sampling of cerebrospinal fluid is associated with a high effort and risk; on the other hand, in the case of a proven genetic defect the diagnosis is considered reliable and does not need to be further confirmed in cases of extreme obesity, given consistent clinical symptoms. Therefore, the detection of α-MSH did not previously play a role in the consideration of extreme obesity. While the failure of the central production of α-MSH as well as defects of the MC4 receptor are clearly associated with early manifest extreme obesity, hardly any data is available on possible clinical effects of the modulation of this appetite regulation mechanism. However, it has now been recognised, in experiments conducted in connection with the present invention, that in extremely overweight people who suffer from a disturbance in the signalling cascade between the formation of leptin in peripheral adipose tissue up to the melanocortin receptor, a reduced α-MSH effect in the hypothalamus contributes causally to obesity. A common feature of all these disturbances would be a reduced central α-MSH production. Thus, a particularly meaningful FAS finding can be generated on the basis of the MSH concentration and in particular on the basis of the α-MSH concentration.

Furthermore, it is possible that in a method according to the invention the at least one first FAS indicator comprises a CLIP concentration in the sample matrix. A CLIP concentration is the concentration of corticotropin-like intermediate peptide (CLIP). The amount of CLIP formed, the amount of which may be equimolar to the α-MSH concentration, allows an indirect measurement of the α-MSH concentration. Accordingly, one is not reliant on the direct measurement of the α-MSH concentration. By measuring the α-MSH concentration, the aforementioned advantages with regard to the desired diagnosis can in turn be achieved. Conclusions regarding the desired FAS finding can also be drawn directly on the basis of the CLIP concentration.

In addition, it is possible that in a method according to the invention the at least one second FAS indicator comprises the concentration of at least one peptide hormone in the sample matrix. By determining the concentrations or the ratios of relevant peptide hormones or at least one peptide hormone, diagnostic conclusions can be drawn regarding the FAS finding. In a similar way as described above with regard to the CLIP concentration, the α-MSH concentration in the blood which is to be determined can be determined indirectly by measuring relevant peptide hormones. The concentration of the at least one peptide hormone is preferably determined by a multiplex method. It can be of further advantage if, in a method according to the invention, the at least one second FAS indicator in each case comprises the concentration of different peptide hormones in the sample matrix. As a result, a high accuracy of the FAS finding can be achieved.

In a method according to the present invention it is moreover possible that in order to generate the FAS finding at least one third FAS indicator which is determined beforehand in the context of a big data analysis is determined which is different from the first FAS indicator and from the second FAS indicator, wherein the at least one third FAS indicator comprises in particular at least one of the following features:
patient's BMI is greater than 35,
patient has anorexia,
patient has grade II-Ill obesity syndrome,
patient shows clinical symptoms of obesity,
indication of disturbance of the functionality of the anorexigenic signal path, FAS, from the patient's genetic background.
   It has transpired, in experiments conducted in connection with the present invention, that the FAS finding can be refined on the basis of a big data analysis, using suitable further FAS indicators, to a degree of precision which was not even remotely conceivable using conventional methods for producing a finding of the generic type. The accuracy of the medical and professional assessment or diagnosis can be increased to a new level on the basis of the in each case larger data volume of measured laboratory values, both in patients with known diagnoses as well as in healthy persons and patients with unknown diagnoses, with statistical and mathematical evaluation using a machine learning method and/or the big data analysis according to the invention. In such cases, the underlying experiences can lead to an overall assessment across all measured values or FAS indicators, with regard to which not only can known questions be taken into account, but previously unknown factual circumstances can also be identified, without pre-existing clinical suspicion or corresponding questions.

In a further embodiment of the present invention it is possible that the at least one first FAS indicator is determined on the basis of at least one measured value of a first analyte in the sample matrix and/or the at least one second FAS indicator is determined on the basis of at least one measured value of a second analyte in the sample matrix, wherein the first analyte and the second analyte are in particular located at different points within a control loop or a synthesis chain. The advantage of this approach is that the functionality of the control loop or the corresponding signal cascade can be measured and detected at different points. The individual analytes are interdependent, that is to say if one analyte is high or low, the other must also be correspondingly high or low. In this way, the desired results can be secured. The analytes, which can be derived from both liquid and dried sample matrices, can be extracted from the sample matrix by immunoprecipitation (IP) or solid phase extraction (SPE). The analytes can be bound to the column material and isolated by in each case selecting a suitable antibody or according to physicochemical properties using a C18 material under reversed phase conditions. Alternatively, other materials such as mixed-mode materials are conceivable and can be used accordingly. A clean extract can be obtained by washing the analytes with aqueous solvents with subsequent elution through organic solvents. The extracted analytes can subsequently be concentrated by evaporation of the organic solvent and reconstituted in an aqueous solution. The extracted analytes can be separated from other matrix components of the sample using reversed phase chromatography in an online LC-MS/MS method. The extracted analytes can also be chemically altered, in particular derivatised. Methods such as Immunoaffinity Chromatography (IAC) and Hydrophilic Interaction Liquid Chromatography (HILIC) are also possible. A mass spectrometric analysis can be performed in a multianalyte method in MRM mode (multiple reaction monitoring), in which specific mass transitions of double- or triple-charged analyte ions can be fragmented in the collision cell of the mass spectrometer and the fragmentions can be detected. By doping stable isotope-labelled internal standards, the quantitative determination of the analyte can be carried out by means of external calibration.

In a method according to the invention it is moreover possible that a plurality of first measured values are determined for a first analyte in the sample matrix and/or a plurality of second measured values are determined for a second analyte in the sample matrix, wherein the first measured values are expanded to form a first group of measured values and/or the second measured values are expanded to form a second group of measured values, wherein the at least one first FAS indicator is determined on the basis of the first group of measured values and the at least one second FAS indicator is determined on the basis of the second group of measured values. With the help of this approach, a dynamic weighting of the FAS indicators is possible, for example in the case of widely deviating FAS indicators. On the basis of the quantitative deviation value that can be determined in this way, a particularly meaningful FAS finding can be generated. According to the invention it is possible that the first measured values are expanded by means of a statistical measure to form a first group of measured values and/or the second measured values are expanded by means of a statistical measure to form a second group of measured values. That is to say, the respective first and second measured values are preferably expanded by means of a mathematical and/or statistical method. In other words, a desired value or indicator in the form of the associated group of measured values can be mathematically formed and interpreted on the basis of the respective first and second measured values. By mathematical operations of individual measured values that are not very meaningful taken on their own, statistical methods can be used to determine meaningful indicators for certain diagnoses. Here, the significance of the calculated indicators can be determined through the statistical evaluation of a large number of measured values in the context of a big data analysis in comparison with persons with known diagnosis.

In a method according to the invention it can also be advantageous if a quantitative mean first deviation value of the first group of measured values from a predefined first reference value and/or a quantitative mean second deviation value of the second group of measured values from a predefined second reference value are determined and the FAS finding is generated as a function of the first deviation value and/or the second deviation value. With the help of this procedure, a relatively accurate FAS finding can be generated.

It can be of further advantage if, in a method according to the present invention, the at least one first FAS indicator, the at least one second FAS indicator and/or the at least one third FAS indicator are multiplied by a weighting factor and the FAS finding is generated as a function of the weighted FAS indicators. As a result, different significances of the FAS indicators can be taken into account, as a result of which an even more meaningful FAS finding can be developed.

In a method according to the invention, a blood sample, a whole blood sample, a plasma sample, a serum sample, a cerebrospinal fluid sample and/or a urine sample, in each case in liquid or dry form, is preferably used as a sample matrix. When using dried sample matrices, the analytes, as explained above, are extracted from the dried sample matrix using suitable extraction agents, wherein a rehydration can be performed with or without organic solvent content. When dried blood is used, an enzymatic cleavage of the analytes can be expanded into peptide fragments. A human plasma sample is preferably used as a sample matrix. Alternatively or in addition, a human blood sample, particularly preferably a human dried blood sample, can also be used.

According to a further aspect of the present invention, a computer program product is provided which is configured and designed to carry out a method as described in detail above on a provided sample matrix. Thus, a computer program product according to the invention brings the same advantages as have been described in detail with reference to the method according to the invention. The computer program product can be implemented as computer-readable instruction code in any suitable programming language, such as JAVA or C++. The computer program product may be stored on a computer-readable storage medium such as a data disk, a removable drive, a volatile or non-volatile memory, or a built-in memory/processor. The instruction code can program a computer or other programmable devices such as a control unit to carry out the desired functions. Furthermore, the computer program product may be provided in a network such as the internet for example, from which it can be downloaded by a user if required. The computer program product can be realised both by means of a computer program, i.e. in software form, and also by means of one or more special electronic circuits, i.e. in hardware form, or in any hybrid form, i.e. by means of software components and hardware components. In addition, storage means with a computer program product according to the invention stored thereon is provided.

A further aspect of the present invention relates to an analysis device for providing a FAS finding for a patient, with a computer program product as described above installed therein, having a determination device for determining at least one first FAS indicator from a sample matrix and for determining at least one second FAS indicator from the sample matrix, wherein the at least one second FAS indicator is different from the at least one first FAS indicator, and a generating device for generating the FAS finding using an indicator spectrum comprising the at least one first FAS indicator and the at least one second FAS indicator. Thus, an analysis device according to the invention also brings the advantages described in detail above.

Further measures to improve the invention are disclosed in the following description of various embodiments of the invention, which are represented schematically in the figures. All features and/or advantages resulting from the claims, the description or the drawing, including constructive details and spatial arrangements, can be essential to the invention both in themselves and in the various combinations.

In each case schematically:

FIG. 1 shows a representation explaining a method according to a first embodiment of the present invention,

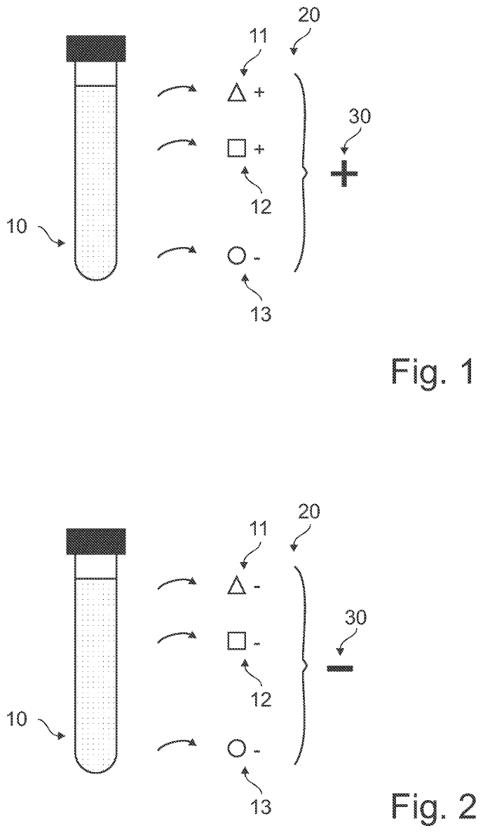

FIG. 2 shows a representation explaining a method according to a second embodiment of the present invention,

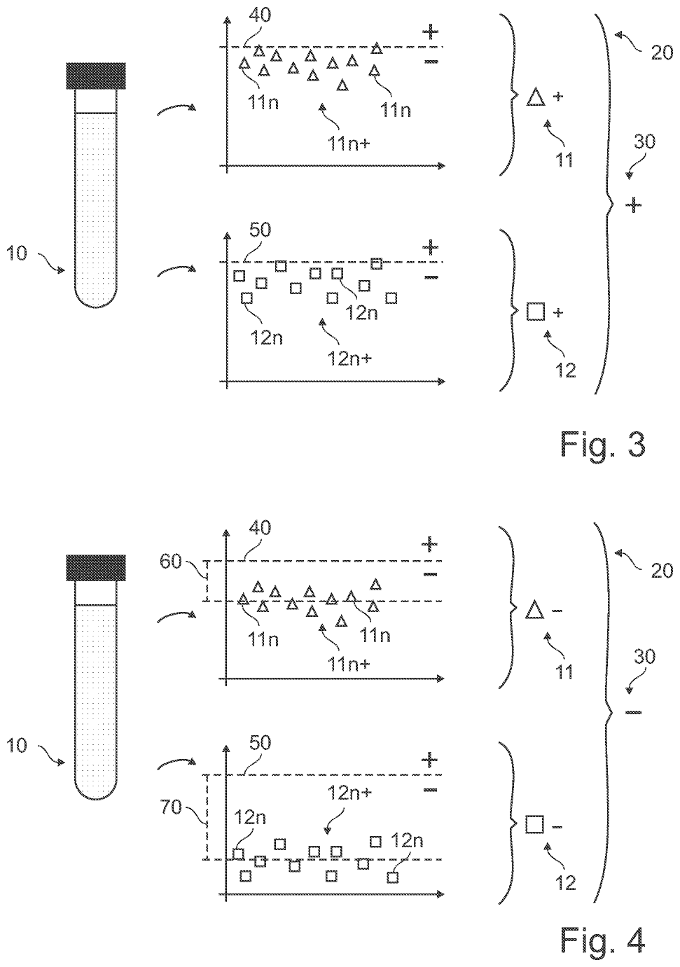

FIG. 3 shows a representation explaining a method according to a third embodiment of the present invention,

FIG. 4 shows a representation explaining a method according to a fourth embodiment of the present invention,

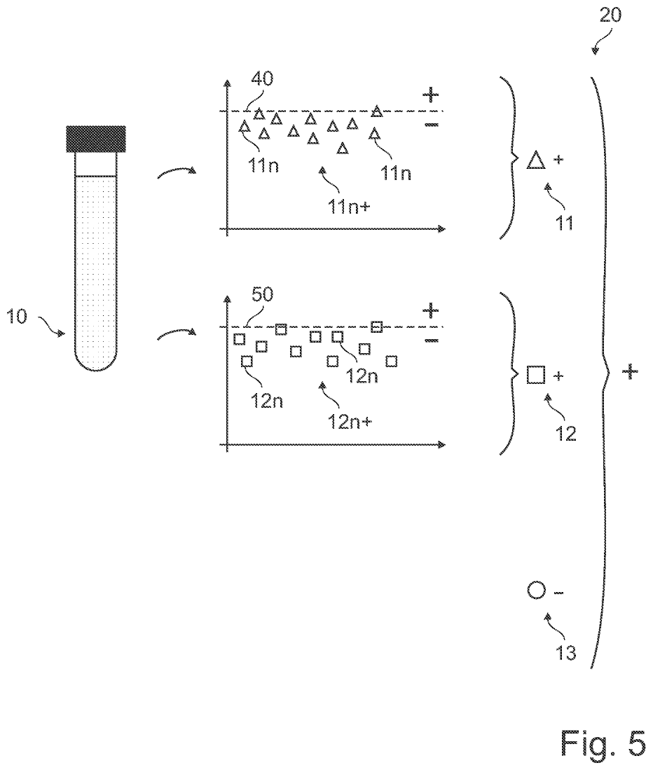

FIG. 5 shows a representation explaining a method according to a fifth embodiment of the present invention,

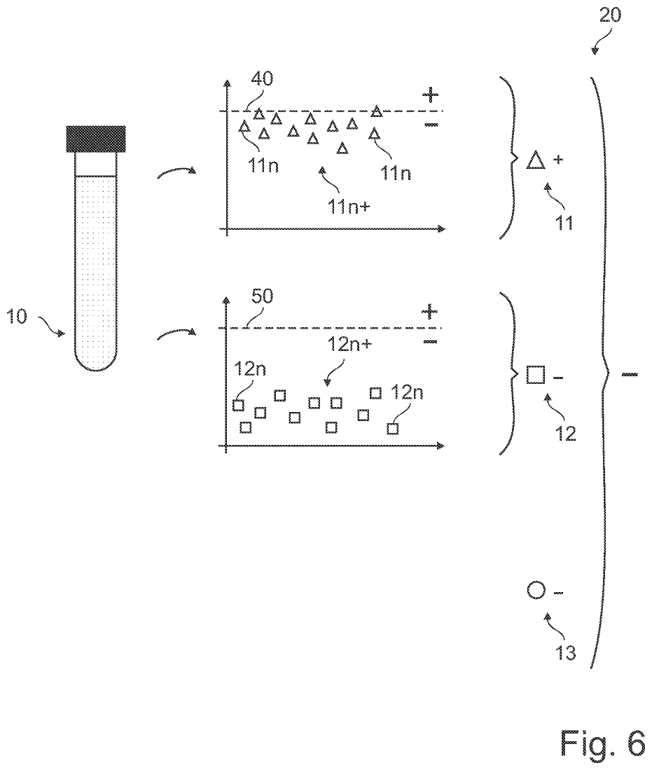

FIG. 6 shows a representation explaining a method according to a sixth embodiment of the present invention,

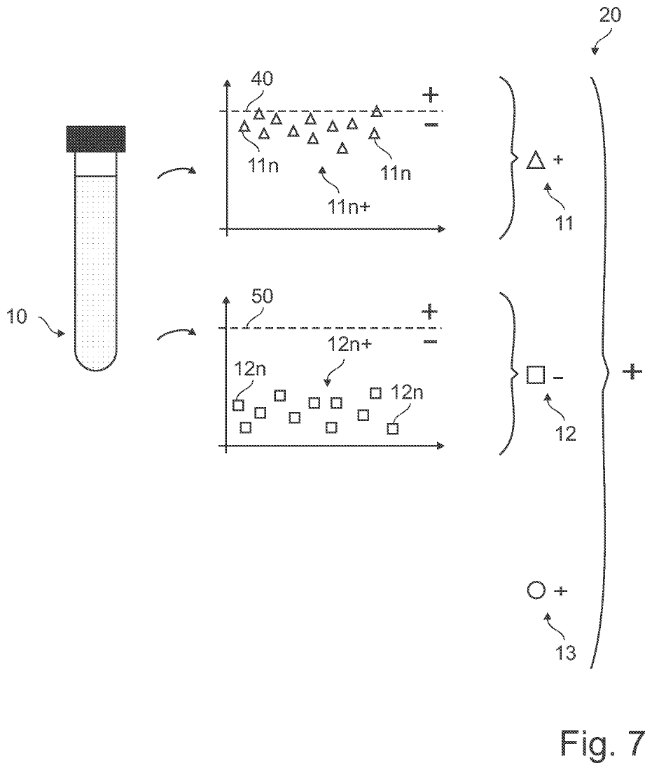

FIG. 7 shows a representation explaining a method according to a seventh embodiment of the present invention, and

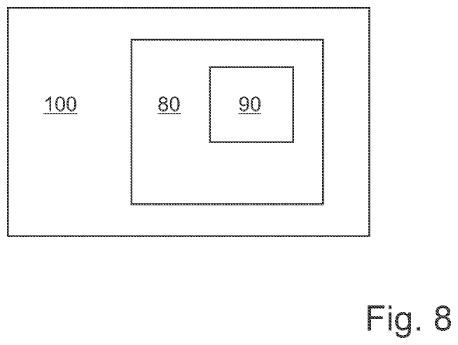

FIG. 8 shows a block diagram representing an analysis device with a storage means and a computer program product stored thereon according to an embodiment of the present invention.

Elements with the same function and mode of action are in each case given the same reference signs in FIGS. 1 to 8.

A method of providing a FAS finding 30 for the functionality of an anorexigenic signal path for a human patient according to a first embodiment is now explained with reference to FIG. 1. FIG. 1 shows a test tube in which a sample matrix 10 in the form of a blood sample of the patient is located. A first FAS indicator 11, a second FAS indicator 12 and a third FAS indicator 13 are now determined from the sample matrix 10, wherein the three FAS indicators 11, 12, 13 are different from each other. The first FAS indicator 11 comprises an α-MSH concentration in the sample matrix 10 and the second FAS indicator 12 comprises a concentration of a peptide hormone in the sample matrix 10.

According to the embodiment shown in FIG. 1, it can be determined on the basis of the first FAS indicator 11 that the functionality of the anorexigenic signal value can be described as normal. The same applies to the second FAS indicator 12. The third FAS indicator indicates that the functionality of the anorexigenic signal value may be disturbed. The three FAS indicators 11, 12, 13 form an indicator spectrum 20. Based on the predominantly positive indication, a FAS finding 30 can now be derived from the overall consideration of the indicator spectrum 20 to the effect that there is probably no, or only a very weakly manifested, malfunction of the anorexigenic signal value. In other words, the anorexigenic signal value appears to function normally or substantially normally. As a result, it can now in turn be concluded that an obesity in question is not, or only scarcely, caused by genetic factors. The illustrated marking of the FAS indicators 11, 12, 13 and of the FAS finding 30 with “+” and “−” also allows exactly the opposite diagnosis, depending on a previously specified interpretation of the sign.

FIG. 2 shows an example according to a second embodiment in which all FAS indicators 11, 12, 13 come up negative, i.e. according to a specified interpretation they indicate a malfunction of the anorexigenic signal value. By considering the associated indicator spectrum 20, a FAS finding 30 can now be derived which indicates a genetic malfunction of the anorexigenic signal value.

A method according to a third embodiment is now explained with reference to FIG. 3. According to the embodiment shown, a plurality of first measured values 11n are determined for a first analyte in the sample matrix 10 and a plurality of second measured values 12n are determined for a second analyte in the sample matrix 10, wherein the first measured values 11n are expanded to form a first group of measured values 11n+ and the second measured values 12n are expanded to form a second group of measured values 12n+. The first FAS indicator 11 is determined on the basis of the first group of measured values 11n+ and the second FAS indicator 12 is determined on the basis of the second group of measured values 12n+. The first analyte and the second analyte are located at different points within a control loop or a synthesis chain.

In particular, the values of the first group of measured values 11n+ are compared with a first reference value 40 and the values of the second group of measured values 12+ are compared with a second reference value. The respective FAS indicator 11, 12 is now concluded on the basis of the respective comparison. Purely by way of example, the reference values 40, 50 shown in FIG. 3 lie above or substantially above the respective group of measured values 11n+, 12n+. Depending on the previous interpretation and definition, the reference values can, alternatively or additionally, also be lower, in particular also below the respective group of measured values, whereby the present result is nevertheless achieved. That is to say, in this case a FAS indicator would lead to a meaningful result even if a group of measured values were above or substantially above a corresponding reference value. Thus, both an increased and a decreased FAS indicator can lead to the present FAS finding 30. Decisive, in particular, is the amount of the distance between the group of measured values and the reference value. This applies to all corresponding figures or associated embodiments.

As above, the first FAS indicator 11 comprises an α-MSH concentration in the sample matrix 10 and the second FAS indicator 12 comprises a concentration of a peptide hormone in the sample matrix 10. Accordingly, the first analyte corresponds to α-MSH and the second analyte to the peptide hormone.

According to FIG. 3, a positive first FAS indicator 11 can be assumed, since the expanded first group of measured values 11n+ is located in a range adjacent to the first reference value 40 and partly above this. Likewise, a positive second FAS indicator 12 can be assumed, since the expanded second group of measured values 12n+ is also located in a range adjacent to the second reference value 50 and partly above this. The FAS finding 30 is consequently also positive. That is to say, in this case a normal or substantially normal functioning or functionality of the anorexigenic signal path can be assumed. However, depending on the specification regarding the interpretation of the respective group of measured values 11n+, 12n+, the result represented in FIG. 3 could also be interpreted to the effect that the first FAS indicator 11 and the second FAS indicator 12 are in each case evaluated negatively, since an insufficient number of measured values 11n, 12n lie above the respective reference value 40, 50. As mentioned above, the overall consideration of the indicator spectrum according to predefined specifications is decisive.

In the fourth exemplary embodiment shown in FIG. 4, a quantitative mean first deviation value 60 of the first group of measured values 11n+ from the predefined first reference value 40 and a quantitative mean second deviation value 70 of the second group of measured values 12n+ from the predefined second reference value 50 are determined, and the FAS finding 30 is generated as a function of the first deviation value 60 and the second deviation value 70. With regard to the first group of measured values 11n+, it can be seen that although it lies relatively close to the first reference value 40, it is not close enough. Therefore, a correspondingly negative value is determined for the first FAS indicator 11. The second group of measured values 12n+ is relatively far from the second reference value 50, for which reason the second FAS indicator is also evaluated negatively. This also results in a negative overall result in the sense of a corresponding FAS finding 30.

Three exemplary embodiments are now explained with reference to FIGS. 5 to 7 in which a third FAS indicator 13, determined beforehand in the context of a big data analysis, which is different from the first FAS indicator 11 and from the second FAS indicator 12, is used to generate the FAS finding 30, wherein the third FAS indicator 13 is the BMI of the patient. According to the fifth embodiment shown in FIG. 5, the third FAS indicator 13 is negative, while the first and the second FAS indicator 11, 12 are in each case positive. The FAS finding 30 is therefore also positive. According to the sixth embodiment shown in FIG. 6, the third FAS indicator 13 is negative, while the first FAS indicator 11 is positive and the second FAS indicator 12 is negative. Due to the predominantly negative FAS indicators, the FAS finding 30 is also negative. According to the seventh embodiment shown in FIG. 7, the third FAS indicator 13 is positive, while the first FAS indicator 11 is positive and the second FAS indicator 12 is negative. Due to the predominantly positive FAS indicators, the FAS finding 30 is also positive in this case. A negative third FAS indicator 13 can be understood to indicate that the patient has an increased or excessively high BMI. As already mentioned above, depending on the definition and interpretation, a negative third FAS indicator 13 can of course also mean exactly the opposite.

FIG. 8 shows an analysis device 100 for providing a FAS finding 30 for a patient, with a computer program product 90 installed therein. The computer program product 90 is stored on a storage means 80 and is configured and designed to carry out a method as described in detail above on a provided sample matrix 10. The analysis device also has a determination device for determining a first FAS indicator 11 from the sample matrix 10 and for determining a second FAS indicator 12 from the sample matrix 10, wherein the second FAS indicator 12 is different from the first FAS indicator 11. In addition, the analysis device has a generating device for generating the FAS finding 30 using the indicator spectrum 20 comprising the first FAS indicator 11 and the second FAS indicator 12.

In addition to the embodiments illustrated, the invention allows for further design principles. Thus, the blood sample can be provided as a liquid blood sample or as a dried blood sample. A whole blood sample, a plasma sample, a serum sample, a cerebrospinal fluid sample and/or a urine sample, in each case in liquid or dry form, can also be used as sample matrix 10. The first FAS indicator 11 can also comprise a CLIP concentration in the sample matrix 10. The second FAS indicator 12 or a plurality of second FAS indicators 12 may in each case comprise the concentration of different peptide hormones in the sample matrix 10. The first FAS indicator 11, the second FAS indicator 12 and/or the third FAS indicator 13 can be multiplied by a weighting factor, wherein the FAS finding 30 is generated as a function of the weighted FAS indicators 11, 12, 13. In general, the first FAS indicator 11 can also be understood as second FAS indicator 12 or third FAS indicator 13, and vice versa. Furthermore, a plurality of first, second and/or third FAS indicators 11, 12, 13 can in each case be determined. The third FAS indicator 13 may comprise features such as an anorexia of the patient, a grade II-III obesity syndrome of the patient, clinical symptoms of obesity in the patient, and/or a direct indication of a disorder in the functionality of the anorexigenic signal path, FAS, in the patient's genetic background.

### LIST OF REFERENCE SIGNS

10 sample matrix
11 first FAS indicator
11n first measured value
11n+ first group of measured values
12 second FAS indicator
12n second measured value
12n+ second group of measured values
13 third FAS indicator
20 indicator spectrum
30 FAS finding
40 first reference value
50 second reference value
60 mean first deviation value
70 mean second deviation value
80 storage means
90 computer program product

1. Method of providing a FAS finding (30) for the functionality of an anorexigenic signal path for a patient, comprising the steps:
providing a sample matrix (10) of a body substance of the patient,
determining at least one first FAS indicator (11) from the sample matrix (10),
determining at least one second FAS indicator (12) from the sample matrix (10), wherein the at least one second FAS indicator (12) is different from the at least one first FAS indicator (11), and
generating the FAS finding (30) using an indicator spectrum (20) comprising the at least one first FAS indicator (11) and the at least one second FAS indicator (12).

2. Method according to claim 1,
characterised in that
the at least one first FAS indicator (11) comprises an MSH concentration, in particular an α-MSH concentration, in the sample matrix (10).

3. Method according to claim 1,
characterised in that
the at least one first FAS indicator (11) comprises a CLIP concentration in the sample matrix (10).

4. Method according to claim 1,
characterised in that
the at least one second FAS indicator (12) comprises the concentration of at least one peptide hormone in the sample matrix (10).

5. Method according to claim 1,
characterised in that the at least one second FAS indicator (12) in each case comprises the concentration of different peptide hormones in the sample matrix (10).

6. Method according to claim 1,
characterised in that
in order to generate the FAS finding (30), at least one third FAS indicator (13) which is determined beforehand in the context of a big data analysis and which is different from the first FAS indicator (11) and is different from the second FAS indicator (12) is determined, wherein the at least one third FAS indicator (13) comprises in particular at least one of the following features:
patient's BMI is greater than 35,
patient has anorexia,
patient has grade II-III obesity syndrome,
patient shows clinical symptoms of obesity,
indication of disturbance of the functionality of the anorexigenic signal path, FAS, from the patient's genetic background.

7. Method according to claim 1,
characterised in that
the at least one first FAS indicator (11) is determined on the basis of at least one measured value (11n) for a first analyte in the sample matrix (10) and/or the at least one second FAS indicator (12) is determined on the basis of at least one measured value (12n) for a second analyte in the sample matrix (10), wherein the first analyte and the second analyte are in particular located at different points within a control loop or a synthesis chain.

8. Method according to claim 1,
characterised in that
a plurality of first measured values (11n) are determined for a first analyte in the sample matrix (10) and/or a plurality of second measured values (12n) are determined for a second analyte in the sample matrix (10), wherein the first measured values (11n) are expanded to form a first group of measured values (11n+) and/or the second measured values (12n) are expanded to form a second group of measured values (12n+), wherein the at least one first FAS indicator (11) is determined on the basis of the first group of measured values (11n+) and the at least one second FAS indicator (12) is determined on the basis of the second group of measured values (12n+).

9. Method according to claim 8,
characterised in that
a quantitative mean first deviation value (60) of the first group of measured values (11n+) from a predefined first reference value (40) and/or a quantitative mean second deviation value (70) of the second group of measured values (12n+) from a predefined second reference value (50) are determined and the FAS finding (30) is generated as a function of the first deviation value (60) and/or the second deviation value (70).

10. Method according to claim 1,
characterised in that
the at least one first FAS indicator (11), the at least one second FAS indicator (12) and/or the at least one third FAS indicator (13) are multiplied by a weighting factor and the FAS finding (30) is generated as a function of the weighted FAS indicators (11, 12, 13).

11. Method according to claim 1,
characterised in that
a blood sample, a whole blood sample, a plasma sample, a serum sample, a cerebrospinal fluid sample and/or a urine sample, in each case in liquid or dry form, is used as the sample matrix (10).

12. Computer program product (90) which is configured and designed to carry out a method according to claim 1 on a provided sample matrix (10).

13. Storage means (80) with a computer program product (90) according to claim 12 stored thereon.

14. Analysis device (100) for providing a FAS finding (30) for a patient, with a computer program product (90) according to claim 12 installed therein, having a determination device for determining at least one first FAS indicator (11) from a sample matrix (10) and for determining at least one second FAS indicator (12) from the sample matrix (10), wherein the at least one second FAS indicator (12) is different from the at least one first FAS indicator (11), and a generating device for generating the FAS finding (30) using an indicator spectrum (20) comprising the at least one first FAS indicator (11) and the at least one second FAS indicator (12).
